SEQUENCE LISTING (36 sequences)

SEQ ID NO: 1 - protein, 221 aa, Homo sapiens
  HLA-H
MVLMAPRTLLLLLSGALALTLTQTWARSHSMRYFYTTMSRPGRGEPRFISVGYVDDTQFVRFDSDDASPREEPRAPWMEREGPEYWDRNTQICKAQAQTERENLRIALRYYNQSEGGSHTMQVMYGCDVGPDGRFLRGYEQHAYDGKDYIALNEDLRSWTAADMAAQITKRKWEAARRAEQLRAYLEGEFVEWLRRYLENGKETLQRADPPQDTYDPPPHL

SEQ ID NO: 2 - protein, 219 aa, Homo sapiens
  HLA-J
MAPRTLLLLLSGTLALAETWAGSHSMRYFSTAVSWPGRGEPSFIAVGYVDDTQFVRVDSDAVSLRMKTRARWVEQEGPEYWDLQTLGAKAQAQTDRVNLRTLLRYYNQSEADPPQDTRDPPPSLNMRHNEVLGSGLLPCGDHIDLAAGWGGPDPGHGARGDQAHRGWNLPEVGGCGSAFWRGTEIHMPCAAQGAAQAPHPEMGHTFLETSRGSKTRRFL

SEQ ID NO: 3 - protein, 246 aa, Homo sapiens
  HLA-L soluble
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPI

SEQ ID NO: 4 - protein, 341 aa, Homo sapiens
  HLA-L membrane-bound
MGVMAPRTLLLLLLGALALTETWAATPVRGWSGGRRGWSRRGWSIGTRRHGTPRATRRFTETCGPCSAITTRARPVTVRLRWQGLHRPERGPALLDRREHSGSDLPAQVGSGQILRAGQGLPEGKCMEWLRRHLENGKETLQHADPPKAHVTQHPISDHEATLRCWALGLYPAEITLTWQQDGEDQTQDTELVETRPAGDGTFQKWVAVVVPSGEEQRYMCHVQHEGLPEPLTLRWEPSSQPTIPIVGIVAGLFLLGAVVTGAVVAAAMWRKKSSGSNCAQYSDASHDTCKEDYACSCSGVCVLISFSPGCPSSLTAAGVIFPVINPTRWKAAPAHRSLWY

SEQ ID NO: 5 - protein, 111 aa, Homo sapiens
  HLA-V
MAPRTLLLLLSGALVLTQTWAGFHSLRYFHTTMSRPGRADPRFLSVGDVDDTQCVRLDSDATSPRMEPRAPWMEQEGPEYWEEETGTAKAKAQFYRVNLRTLSGYYNQSEA

SEQ ID NO: 6 - protein, 206 aa, Homo sapiens
  HLA-Y
MAVVAPRTLLLLLSGALALTQTWAGSHSMRYFSTSVSRPGSGEPRFIAVGYVDDTQFVRFDSDAASQRMEPRAPWMEQEEPEYWDRQTQISKTNAQIDLESLRIALRYYNQSEAGSHTIQRMSGCDVGSDGRFLRGYRQDAYDGKDYIALNEDLRSWTAADMAAQITQRKWEAARQAEQLRAYLEGECMEWLRRYLENGKETLQRT

SEQ ID NO: 7 - protein, 357 aa, Homo sapiens
  HLA-E
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHS

SEQ ID NO: 8 - protein, 442 aa, Homo sapiens
  HLA-F1
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAAYSVVSGNLMITWWSSLFLLGVLFQGYLGCLRSHSVLGRRKVGDMWILFFLWLWTSFNTAFLALQSLRFGFGFRRGRSFLLRSWHHLMKRVQIKIFD

SEQ ID NO: 9 - protein, 346 aa, Homo sapiens
  HLA-F2
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 10 - protein, 254 aa, Homo sapiens
  HLA-F3
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRAEQSPQPTIPIVGIVAGLVVLGAVVTGAVVAAVMWRKKSSDRNRGSYSQAAV

SEQ ID NO: 11 - protein, 337 aa, Homo sapiens
  HLA-G1
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 12 - protein, 245 aa, Homo sapiens
  HLA-G2
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 13 - protein, 129 aa, Homo sapiens
  HLA-G3
MVVMAPRTLFLLLSGALTLTETWASHSMRYFSAAVSRPGRGEPRFIAMGYVDDTQFVRFDSDSACPRMEPRAPWVEQEGPEYWEEETRNKQSSLPTIPIMGIVAGLVVLAAVVTGAAVAAVLWRKKSSD

SEQ ID NO: 14 - protein, 245 aa, Homo sapiens
  HLA-G4
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRAKQSSLPTIPIMGIVAGLVVLAAVVTGAAVAAVLWRKKSSD

SEQ ID NO: 15 - protein, 318 aa, Homo sapiens
  HLA-G5
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 16 - protein, 226 aa, Homo sapiens
  HLA-G6
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 17 - protein, 115 aa, Homo sapiens
  HLA-G7
MVVMAPRTLFLLLSGALTLTETWASHSMRYFSAAVSRPGRGEPRFIAMGYVDDTQFVRFDSDSACPRMEPRAPWVEQEGPEYWEEETRNTKAHAQTDRMNLQTLRGYYNQSEASE

SEQ ID NO: 18 - DNA, 1704 bp, Homo sapiens
  HLA-H
agtttctcttcttctcacaacctgcgacgggtccttcttccttgatactcacgaagcggacacagttctcattcccactaggtgtcgggtttctagagaagccaatcggtgccgccgcggtcccggttctaaagtccccacgcacccaccgggactcagattctccccagacgccgaggatggtgctcatggcgccccgaaccctcctcctgctgctctcaggggccctggccctgaccctgacccagacctgggcgcgctcccactccatgaggtatttctacaccaccatgtcccggcccggccgcggggagccccgcttcatctccgtcggctacgtggacgatacgcagttcgtgcggttcgacagcgacgacgcgagtccgagagaggagccgcgggcgccgtggatggagcgggaggggccagagtattgggaccggaacacacagatctgcaaggcccaggcacagactgaacgagagaacctgcggatcgcgctccgctactacaaccagagcgagggcggttctcacaccatgcaggtgatgtatggctgcgacgtggggcccgacgggcgcttcctccgcgggtatgaacagcacgcctacgacggcaaggattacatcgccctgaacgaggacctgcgctcctggaccgcggcggacatggcagctcagatcaccaagcgcaagtgggaggcggcccgtcgggcggagcagctgagagcctacctggagggcgagttcgtggagtggctccgcagatacctggagaacgggaaggagacgctgcagcgcgcggaccccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccgagcccctcaccctgagatgggagccatcttcccagcccaacgtccccatcgtgggcatcgttgctggcctggttctacttgtagctgtggtcactggagctgtggtcgctgctgtaatgtggaggaagaagagctcagatagaaaaggagggagctactctcaggctgcaacagcaacagtgcccagggctctgatgtgtctctcacggcttgaaagtgtgagacagctgccttgtgtgggactgagaggcaagagttgttcctgccttccctttgtgacttgaagaaccctgactttctttctacaaaggcacctgaatgtgtctgtgttcctgtaggcataatgtgtggaggaggggagaccaacccaccctcatgtccaccatgaccctcttccccacgctgatctgtgttccctccccaatcatctttcctgttccagagaggaggggctgagatgtctccatctttttctcaactttatgtgcactgagctgtaacttcttacttccctcttaaaattagaatctgagtaaacatttactttttcaaattcttgccatgagaggttgatgacttaattaaaggagaagattcctaaaatttgagagacaaaataaatggaacacatgagaaccttccag

SEQ ID NO: 19 - DNA, 1552 bp, Homo sapiens
  HLA-J
ctatactatctcatgcacccaggcacaacttttccagatttaaagaaaaagaaaaaagaaataaaagaaaaaaacctctgtctctacacctccattcccagggagagctccctctctggcaccaagctccctggggtgagttttctttttgaagagtccaggggaacagcctgcgacgggtccttcttcctggacactcacgacgcggacccagttctcactcccactgagtgtcgggttttagggaagccaatcagcgtcgcgcggccccggttctaaagtccccacgcacccaccgggactcggagtctccccagacgccgacgatggggtcatggcgccccgaaccctcctcctgctgctctcggggaccctggccctggccgagacctgggcgggctcccactccatgaggtatttcagcaccgccgtttcctggccgggccgcggggagcccagcttcattgccgtgggctacgtggacgacacgcagttcgtgcgggtcgacagtgacgccgtgagtctgaggatgaagacgcgggcgcggtgggtggagcaggaggggccggagtattgggacctacagacactgggcgccaaggcccaggcacagactgaccgagtgaacctgcggaccctgctccgctactacaaccagagcgaggcggaccccccccaagacacacgtgacccacccccctctctgaacatgaggcataacgaggtcctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggctgcccaagcccctcatcctgagatgggtcacacatttctggaaacttctcaaggttccaagactaggaggttcctctaggacctcatggccctgctaccttcctggcctctcacaggacgttttcttcccgcagatagaaaaggagggagctactctcaggctgcaagcagccaaagtgcccagggctctgatgtgtctctcacggcttgtaaagtgtgagacagctgccttgtgtgggactgagaggcaagatttgttcatgccttccctttgtgacttcaagaaccctgacttctctttctgcaaaggcatctgaatgtgtctgtgtccctataggcataatgtgaggtggtggggagaccagcccacacccgtgtccaccatgaccctgttccccacactgacctacattccttccccgatcacctttcctgttccagagaagtggtgctgggatgtctccatctctgtctcaacttcatggtgcactgagctgtaacttcttacttccctattaaaattagaatctgagtataaatttacttttttcaaattatttccatgacgggttgatgggttaattaaaggagaagattcctaaaatttgagagacaaaataaatggaagacatgagaa

SEQ ID NO: 20 - DNA, 898 bp, Homo sapiens
  HLA-L soluble
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcag

SEQ ID NO: 21 - DNA, 4622 bp, Homo sapiens
  HLA-L membrane-bound
acgatcccggcactacagtcccggcgcaaccacccgcactcagattctccccaaacgccaaggatgggggtcatggctccccgaaccctcctcctgctgctcttgggggccctggccctgaccgagacctgggccgcgactccgtgagtccgaggatggagcggcgggcgccgtgggtggagcaggaggggctggagtattgggaccaggagacacggaacgccaagggccacgcgcagatttaccgagtgaacctgcggaccctgctccgctattacaaccagagcgaggccggtatgaacagttcgcctacgatggcaaggattacatcgccctgaacgaggacctgcactcctggaccgccgcgaacacagcggctcagatctcccagcacaagtgggaagcggacaaatactcagagcaggtcagggcctacctgagggcaagtgcatggagtggctccgcagacacctggagaacgggaaggagacgctgcagcacgcggatcccccaaaggcacatgtgacccagcaccccatctctgaccatgaggccaccctgaggtgctgggccctgggcctctaccctgcggagatcacactgacctggcagcaggatggggaggaccagacccaggacacggagcttgtggagaccaggcctgcaggggacggaaccttccagaagtgggtggctgtagtggtgccttccggagaggagcagagatacatgtgccatgtgcagcatgaggggctgccagagcccctcaccctgagatgggagccgtcttctcagcccaccatccccatcgtgggcatcgttgctggcctgtttctccttggagctgtggtcactggagctgtggttgctgctgcgatgtggaggaagaaaagctcaggcagcaattgtgctcagtactctgatgcatctcatgatacttgtaaagaggactatgcctgttcctgttctggtgtctgcgttctgatctctttctcccctgggtgtccctcatctctgacagcagcaggagtcatttttcctgtcattaaccccacaaggtggaaggcagcccctgcacacagaagtctgtggtattaagagatgaattttcaagcccgtgcagcttttaccctatttccagggctctttcttggattgtattttctatcttttccccaacctttttaaaggaactagattctgaaattagcagagaagagggatgccacaagttctcatcttaggtaactttctagtggaactcctcttctgctcagctctcctacccactctcccttccctgagttgtagtaatcctagcactggctctaatgcaaactcatggatctataaagcaaagtctaacttagatttatatttgtttggaaattgggattcatagtcaaagattgttctttcctaagagggaaatataattgcatgctgcagtgtgcagagggttggtgtgaaggagggatgcagggaggaagggagggaggacacacaagcagcactgctgggaaaagcacaggcggcctggatgtcagtgtgaggggaccttgtgctgtcgttgctgcaaaaccgcatttggcctgaggctatgttaataaagatactgcctttagaataggaggtgctctacagtgatgattcattcagccgacatttgctgtctgccagacatatgacagaatgtttttgcatctggggaaagtcattgaagtaaaatcagaaaaatctctagccttgtggagcatgtgttccagtgggaagaggcagacggtacatacactctaatatatgcagagtaaatgaggaaagtgttagaaggtgataagtgctgtggaacaggtgatcagagtatgggttgtgggacagagaaggtagctattgtgccggggttgtcagcgtgggccttgttgggaaggtgacctttgatgaaatatttgaaggacataaaggaatttgtcatgagggtatctggaagaagttttttctagggagtaggaaccttcagtgtcagtgtaccagggcaggatcatgtctgtgtgttctgggaagaacacgggatcgggtatggctagagcagagagtcactgagataaggtcaggggtttggtcagatcatgtgggcatagggctcaagtatgtgggaaggattttgattttgaatgagatagttttaagcagaataaagacatgccacaacttctcttttaaaaggatcactgtagctgctctgctgagaacagaatccaaaggccggcgatgagcaaggcaggtgggaaaactgtaggaaatgagtgcagtatttcaggctggagatgtcggttacttcaactggggtgtgagcagtggaaatagtgggacgtgattggattcctactatttccaatcactttataccgcattttctaatggactaaatctggggtatgagaaagaagagtaaaggataccaaaaatgtcagactgtgactaaaaagagttgccatcagctgagaatgagaagactagcaggagcatatgagaggaggggacgtcgcaggcagtcactatgggagacgtgggatctgagatgccgctgagaaataccagtgaggtagtcgggttggcagttggacagatgaatctggagacatttaggagaaatagacttgggaggtgatgtcatataacagttatttaaagccttgagtctgaatgacgtctccaagggagtgattggctgtagaagagaacaggaacaaggactgaacactaggcctctgttgctaaaggatctgatcagacaacacacctagatcagactgcacagtcctgaccccacatctagaaggtacatagaccagggagttctagactttcctgtggacaggaatcacctggacatcaccttaagtctaagctgatctggaatcgagaatgagatttcctacttatataatgttgctgttggcgctgatgctgctggtcttcagatcccacttttggtagcaagaacacagaccaggattcctaggctatgcatcagcctcgcctgtgaggcttgttaataagcaattcctgcactccatgcgcaacattctgacacaggggcatctgtggagaggcctgagtattctacaacaagcccacagcaaacctggtgctcagccagatttgatatcactgagatcagtagttggagaatgcccaggatggggaggggtctcagacccacatttaagtgttgctttattctgggttttttatttatttatttatttatttttaaggaggatgtgtttctttaattataagacaggatgctgagagataaatgtcattttctctatcatggggtatagccagatggaagattgagaagtggctcacagctcagcagaatgaaaaaatatctgaatgctgctttctgaaactactctccagaatgatttcacactcactccttggagcaaacaatgacttgcaaatttttctaatttaaacataaaggagtgtacatattggtattagtattcattttattttggggaagggcactgtattagtccatagtccgttttcacactgccgataaagacatacccaacattgggaagaaaaagaggtttaattggacttacagttccatttggctggggaggcctcagaatcatggtgggaggcgaaaggcacttcttacatggtggtggcaagagaaaatgaggaagaagcaaatgccaaaacccctgataaacacattggatctcaggagacttattcattatcatgagaatagcatgggaaagactggcccccatgattcaattacctccccctgggtccctcccacaacatgtgggaattctgggagatacaattcaagttgagatttgggtggggacacagccaaaccacattggacacagaaccaggtttgaagctacacagccaggaacataatccacagccaccctaattcagatctctcataggaaccactgtccctgctcctgagcacagatgctactgcatatacctctgataccctgatggccgacactgggccctgtggcaaagactgctatcactgctgctcctgagaactgctccactactgctcctcagccatctttaccaaaatgcagtatttactgtcccagcctctctgtgtcatctcatcctgattagaagcccacatgtggttatctaaattgtgcagccaaagcctcttgcagtgtttaactgcaataatgttggggaaagtgaatttttctcctttgtagaaggaggtagtccctgccttctaataagactcttcaacataggaagagaattcagttgctggaggtagaggggtgagggatggaaaaagaatgacaaatttcaattcctagaatcatgttctgagactagaactttatctagtacattgcaggcacctgggtttggttgagtgtataataaatgacatagttcaacttattcccttgacagtttgttttggggtccagcttttgtctaccccagttttcacacacagatacgtggagaagcattgtgtgatggtaaaatgtttacttgaaagcctttttccctatctttgtctcttgctaggattaaaaacccgtatctgt

SEQ ID NO: 22 - DNA, 1285 bp, Homo sapiens
  HLA-V
gaaacattgagacagagcgcttggcacagaagtagcggggtcagggcgaagtcccagggcctcaggcgtggctctcaggatctcaggccccaaaggcggtgtatggattggggaggcccagcgctgggcattccccatctttgcagggtttctcttctccctctcccaacctgtgtcgggtccttcttcctgggtactcaccgggctgccccagttctcactcccattgagtgtcgggtttctagagaagccaatcaatgtagccgcggtcccggttctaaagttcccacgcacccaccgggactccgattcttcccagtcgccgaggatggtgtcatggcgccccgaaccctgcttctgctgctctcgggggccctggtcctgacccagacctgggcaggcttccactccttgaggtatttccacaccaccatgtcccggcccggccgcgcggatccccgcttcctctccgtgggcgacgtggacgacacgcagtgcgtgcggctcgacagcgacgccacgagtcccaggatggagccgcgggcgccgtggatggagcaggaggggccggaatattgggaagaggagacagggaccgccaaggccaaagcacagttttaccgagtgaacctgcggaccctgagcggctactacaaccagagtgaggcctaagtgcagcttcattccctccctgttcgtgtggcctggacttaatgactcacttctaactgatagagtaatgctgacataatagtttgtgattctgggtgtagaacataagactcactgaagtttctactttggttctttctttctctggaatcatgagccctgggggaagctggctgttgtgtcataaggaggcctgtggtccatgtgactaggaagtgagtcctcctgggaccagacaataagaagctaaagcctcttccaaaagccatgtgagagattcttgtgtcttgtgaatccccggccccatttgagccctcagatgattcagccctggaagacaactagactgcaacgttgtgagaggccctgagccagaagcattcagagaaacttctcctggattcctgaccatggataactgtgggagatgataaatatttgttgatttgagctgctaagttgtaggtgacttgttatgcagcagtagataactaatacagcttcacaagagaggatgaatcactgaactttttcatttgctctaaattcattataagatattaaacatgtcatttgcttttaatatttaataaaaatttccatggctatataa

SEQ ID NO: 23 - DNA, 1095 bp, Homo sapiens
  HLA-Y
atggcggtcgtggcgccccgaaccctcctcctgctactctcgggggccctggccctgacccagacctgggcgggctcccactccatgaggtatttctccacatccgtgtcccggcccggcagtggagagccccgcttcatcgcagtgggctacgtggacgacacgcagttcgtgcggttcgacagcgacgccgcgagccagaggatggagccgcgggcgccgtggatggagcaggaggagccggagtattgggaccggcagacacagatctccaagaccaacgcacagattgacctagagagcctgcggatcgcgctccgctactacaaccagagcgaggccggttctcacaccatccagaggatgtctggctgcgacgtggggtcggacgggcgcttcctccgcgggtaccggcaggacgcctacgacggcaaggattacatcgccctgaacgaggacctgcgctcttggaccgcggcggacatggcggctcagatcacccagcgcaagtgggaggcggcccgtcaggcggagcagttgagagcctacctggagggcgagtgcatggagtggctccgcagatacctggagaacgggaaggagacgctgcagcgcacggacccccccccaagacacatatgacccaccaccccatctctgaccatgaggccaccctgaggtgctgggccctgagcttctaccctgcggagatcacactgacctggcagcgggatggggaggaccagacccaggacacggagctcgtggagaccaggcctgcaggggatggaaccttccagaagtgggcgtctgtggtggtgccttctggacaggagcagagatacacctgccatgtgcagcatgagggtctgcccaagcccctcaccctgagatgggagccgtcttcccagcccaccatccccatcgtgggcatccttgctggcctggttctctttggagctgtgatcgctggagctgtggtcgctgctgtgatgtggaggaggaagagctcagatagaaaaggagggagctactctcaggctgcaagcagtgacagtgcccagggctctgatgtgtctctcacagcttgtaaag

SEQ ID NO: 24 - DNA, 2548 bp, Homo sapiens
  HLA-E
ctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaagaacctggatctcaatttaatttttcatattcttgcaatgaaatggacttgaggaagctaagatcatagctagaaatacagataattccacagcacatctctagcaaatttagcctattcctattctctagcctattccttaccacctgtaatcttgaccatataccttggagttgaatattgttttcatactgctgtggtttgaatgttccctccaacactcatgttgagacttaatccctaatgtggcaatactgaaaggtggggcctttgagatgtgattggatcgtaaggctgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccagggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgaggccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaactttgaagtaaaataaaagctgtgtttgagcatca

SEQ ID NO: 25 - DNA, 1480 bp, Homo sapiens
  HLA-F1
atatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagcctactcagtggtcagcggaaacttgatgataacatggtggtcaagcttatttctcctgggggtgctcttccaaggatatttgggctgcctccggagtcacagtgtcttgggccgccggaaggtgggtgacatgtggatcttgttttttttgtggctgtggacatctttcaacactgccttcttggccttgcaaagccttcgctttggcttcggctttaggaggggcaggagcttccttcttcgttcttggcaccatcttatgaaaagggtccagattaagatttttgactgagtcattctaaagtaagttgcaagacccatgatactagaccactaaatacttcatcacacacctcctaagaataagaaccaacattatcacaccaaagaaaataaataattccataatatta

SEQ ID NO: 26 - DNA, 1301 bp, Homo sapiens
  HLA-F2
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 27 - DNA, 912 bp, Homo sapiens
  HLA-F3
atatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttataga

SEQ ID NO: 28 - DNA, 1578 bp, Homo sapiens
  HLA-G1
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 29 - DNA, 1302 bp, Homo sapiens
  HLA-G2
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 30 - DNA, 1026 bp, Homo sapiens
  HLA-G3
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 31 - DNA, 1302 bp, Homo sapiens
  HLA-G4
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 32 - DNA, 1138 bp, Homo sapiens
  HLA-G5
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 33 - DNA, 862 bp, Homo sapiens
  HLA-G6
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccaacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 34 - DNA, 529 bp, Homo sapiens
  HLA-G7
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagtgagtaa

SEQ ID NO: 35 - protein, 5 aa, Artificial Sequence
  GS-linker
GGGGS

SEQ ID NO: 36 - protein, 4 aa, Artificial Sequence
  redox-active site
CGPC

(19) United States
(12) Patent Application Publication
(10) Pub. No.: US 2022/0380848 A1
(43) Pub. Date: Dec. 1, 2022

(54) HLA CLASS I MOLECULES IN IN VITRO FERTILIZATION AND FURTHER MEDICAL IMPLICATIONS

(71) Applicant: Intellexon GmbH, Pöcking (DE)

(72) Inventors: Wolfgang WÜRFEL, Odelzhausen (DE); Ralph Markus WIRTZ, Köln (DE); Christoph WINTERHALTER, Pöcking (DE); Franziska WÜRFEL, Odelzhausen (DE)

(21) Appl. No.: 17/771,662

(22) PCT Filed: Oct. 19, 2020
(86) PCT No.: PCT/EP2020/079347

§ 371 Date: Apr. 25, 2022

(30) Foreign Application Priority Data
Oct. 25, 2019 (EP) 19205450.0

(51) Int. Cl. C12Q 1/6881; A61K 31/713; A61K 38/17; C07K 14/74

(52) U.S. Cl. CPC C12Q 1/6881; A61K 31/713; A61K 38/1774; C07K 14/70539

(57) ABSTRACT

The present invention relates to a nucleic acid molecule, a vector, a host cell, or a protein or peptide, or any combination thereof for use in a method of increasing efficiency of embryonic implantation in an in vitro fertilization programme, (I) wherein the at least one nucleic acid molecule is selected from nucleic acid molecules (a) encoding a polypeptide comprising or consisting of the amino acid sequence of any one of SEQ ID NOs 1 to 17, (b) comprising or consisting of the nucleotide sequence of any one of SEQ ID NOs 18 to 23, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and (II) the vector comprises the nucleic acid molecule of (I); (III) the host cell is transformed, transduced or transfected with the vector of (II); and (IV) the at least one protein or peptide is selected from proteins or peptides being encoded by the nucleic acid molecule of (I); and wherein the method of increasing embryonic implantation efficiency comprises (i) contacting the nucleic acid molecule, vector, host cell, or protein or peptide, or any combination thereof with the unfertilized, fertilized oocyte, and/or preimplantation embryo prior to the transfer of the fertilized oocyte or preimplantation embryo to the uterus; or (ii) contacting the nucleic acid molecule, vector, host cell, or protein or peptide, or any combination thereof with the uterus prior to, simultaneously with and/or after the transfer of the fertilized oocyte or preimplantation embryo to the uterus; or (iii) systemically administering the nucleic acid molecule, vector, host cell, or protein or peptide, or any combination prior to, simultaneously with and/or after the transfer of the fertilized oocyte or preimplantation embryo to the uterus, preferably via injection, transdermal and/or vaginal administration.

The present invention relates to a nucleic acid molecule, a vector, a host cell, or a protein or peptide, or any combination thereof for use in a method of increasing efficiency of embryonic implantation in an in vitro fertilization programme, (I) wherein the at least one nucleic acid molecule is selected from nucleic acid molecules (a) encoding a polypeptide comprising or consisting of the amino acid sequence of any one of SEQ ID NOs 1 to 17, (b) comprising or consisting of the nucleotide sequence of any one of SEQ ID NOs 18 to 23, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and (II) the vector comprises the nucleic acid molecule of (I); (III) the host cell is transformed, transduced or transfected with the vector of (II); and (IV) the at least one protein or peptide is selected from proteins or peptides being encoded by the nucleic acid molecule of (I); and wherein the method of increasing embryonic implantation efficiency comprises (i) contacting the nucleic acid molecule, vector, host cell, or protein or peptide, or any combination thereof with the unfertilized, fertilized oocyte, and/or preimplantation embryo prior to the transfer of the fertilized oocyte or preimplantation embryo to the uterus; or (ii) contacting the nucleic acid molecule, vector, host cell, or protein or peptide, or any combination thereof with the uterus prior to, simultaneously with and/or after the transfer of the fertilized oocyte or preimplantation embryo to the uterus; or (iii) systemically administering the nucleic acid molecule, vector, host cell, or protein or peptide, or any combination prior to, simultaneously with and/or after the transfer of the fertilized oocyte or preimplantation embryo to the uterus, preferably via injection, transdermal and/or vaginal administration.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The human leukocyte antigen (HLA) system or complex is a gene complex encoding the major histocompatibility complex (MHC) proteins in humans. The HLA gene complex resides on a 3 Mbp stretch within chromosome 6p21. Human leukocyte antigen (HLA) genes have a long research history as important targets in biomedical science and treatment. More than 100 diseases have been associated with different alleles of histocompatibility complex genes. It is meanwhile fifty years since the first description of an association between HLA and human disease. HLA molecules have proven to be central to physiology, protective immunity and deleterious, disease-causing autoimmune reactivity (Debdrou et al. (2018), Nature Reviews Immunology volume 18, pages 325-339).

The role of HLA genes in autoimmune diseases is reviewed, for example, in Gough and Simmonds, Curr Genomics. 2007 November; 8(7): 453-465. For instance, the HLA-B27 allele increases the risk of developing an inflammatory joint disease called ankylosing spondylitis.

Many other disorders involving abnormal immune function and some forms of cancer have also been associated with specific HLA alleles. For example, EP 2 561 890 describes a procedure for an immunological treatment of cancer. The patent describes, in a first step, the profiling of the non-classical HLA class Ib group in primary tumor tissue as well as metastases and recurrent tumors. The second step comprises tailored antibody therapies.

The HLA class Ib genes, HLA-E, HLA-F, and HLAG, were discovered long after the classical HLA class Ia genes. The elucidation of their functions had a modest beginning. However, their basic functions and involvement in pathophysiology and a range of diseases are now emerging. Although results from a range of studies support the functional roles for the HLA class Ib molecules in adult life, especially HLA-G and HLA-F have been studied in relation to reproduction and pregnancy. The expression of HLA class Ib proteins at the feto-maternal interface in the placenta seems to be important for the maternal acceptance of the semi-allogeneic foetus (Persson et al. (2017), Immunogenetics, DOI 10.1007/s00251-017-0988-4). In pregnancy, the placenta, more specifically the trophoblast, creates an “interface” between the embryo/foetus and the maternal immune system. Trophoblasts do not express the “original” HLA identification of the embryo/foetus (HLA-A to -DQ), but instead show the non-classical HLA Ib group E, F, and G. During interaction with specific receptors of NK cells (e.g., killer-immunoglobulin-like receptors (KIR)) and lymphocytes (lymphocyte-immunoglobulin-like receptors (LIL-R)), the non-classical HLA groups inhibit these immunocompetent cells outside pregnancy (Würfel et al. (2019), Int. J. Mol. Sci. 2019, 20, 1830; doi:10.3390/ijms20081830).

Despite all the knowledge which has already been gathered on the association of HLA genes and diseases, there is still a need to focus research on the HLA genes and in particular to identify further targets for biomedical science and treatment based on the HLA system. This need is addressed by the present invention.

Hence, the present invention relates in first aspect to a nucleic acid molecule, a vector, a host cell, or a protein or peptide, or any combination thereof for use in a method of increasing efficiency of embryonic implantation in an in vitro fertilization programme, (I) wherein the at least one nucleic acid molecule is selected from nucleic acid molecules (a) encoding a polypeptide comprising or consisting of the amino acid sequence of any one of SEQ ID NOs 1 to 17, (b) comprising or consisting of the nucleotide sequence of any one of SEQ ID NOs 18 to 34, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and (II) the vector comprises the nucleic acid molecule of (I); (III) the host cell is transformed, transduced or transfected with the vector of (II); and (IV) the at least one protein or peptide is selected from proteins or peptides being encoded by the nucleic acid molecule of (I); and wherein the method of increasing embryonic implantation efficiency comprises (i) contacting the nucleic acid molecule, vector, host cell, or protein or peptide, or any combination thereof with the unfertilized, fertilized oocyte, and/or preimplantation embryo prior to the transfer of the fertilized oocyte or preimplantation embryo to the uterus; or (ii) contacting the nucleic acid molecule, vector, host cell, or protein or peptide, or any combination thereof with the uterus prior to, simultaneously with and/or after the transfer of the fertilized oocyte or preimplantation embryo to the uterus; or (iii) systemically administering the nucleic acid molecule, vector, host cell, or protein or peptide, or any combination prior to, simultaneously with and/or after the transfer of the fertilized oocyte or preimplantation embryo to the uterus, preferably via injection, transdermal and/or vaginal administration.

The present invention also relates to a method of increasing efficiency of embryonic implantation in an in vitro fertilization programme comprising (i) contacting a nucleic acid molecule, vector, host cell, or protein or peptide, or any combination thereof with the unfertilized, fertilized oocyte, and/or preimplantation embryo prior to the transfer of the fertilized oocyte or preimplantation embryo to the uterus; or (ii) contacting a nucleic acid molecule, vector, host cell, or protein or peptide, or any combination thereof with the uterus prior to, simultaneously with and/or after the transfer of the fertilized oocyte or preimplantation embryo to the uterus; or (iii) systemically administering a nucleic acid molecule, vector, host cell, or protein or peptide, or any combination prior to, simultaneously with and/or after the transfer of the fertilized oocyte or preimplantation embryo to the uterus, preferably via injection, transdermal and/or vaginal administration, wherein the nucleic acid molecule, vector, host cell, or protein or peptide, or any combination thereof is as defined in connection with the first aspect of the invention.

An in vitro fertilization (IVF) programme is a complex series of steps used to help females with fertility problems, for example, to prevent genetic problems or to assist with the conception of a child. An IVF programme comprises the steps of (i) collecting a mature unfertilized oocyte from ovaries of a donor female, (ii) fertilization of the isolated mature unfertilized oocyte with sperm to obtain a fertilized ooyte, (iii) optionally in vitro preimplantation development of the fertilized ooyte into a preimplantation embryo, and (iv) transfer of the fertilized ooyte or the preimplantation embryo to the uterus of a female. One full cycle of an IVF programme generally takes about three weeks. Mature oocytes are characterized in that they can be fertilized by sperm. Mature ooytes are generally characterized by an adjacent polar body. The procedure can be done using the own ooycyte of the female to be treated by IVF and the partner's sperm. IVF may also involve oocytes, sperm or preimplantation embryos from a known or anonymous donor. In vivo, during normal pregnancy the fertilized ooyte develops into a preimplantation embryo in the ampulla of the oviduct. However, this development can also take place in vitro. Hence, either a fertilized ooyte or a preimplantation embryo can be transferred into the uterus of a female. IVF is currently the most effective form of assisted reproductive technology.

The method of the first aspect of the invention comprises in case of an unfertilized ooyte as is referred to in item (i) above, the fertilization of the unfertilized ooyte by sperm and optionally the further development into a preimplantation embryo prior to the transfer of the fertilized ooyte or preimplantation embryo into the uterus.

Embryonic implantation is the stage of pregnancy at which the embryo adheres to the wall of the uterus. At this stage of prenatal development, the conceptus is called a blastocyst. It is by this adhesion that the embryo receives oxygen and nutrients from the mother to be able to grow. In humans during normal pregnancy, implantation of the preimplantation embryo is most likely to occur around nine days after ovulation; however, this can range between 6 and 12 days. Also in an IVF programme the fertilized oocyte or preimplantation embryo adheres to the wall of the uterus after it has been placed into the uterus.

The female to be treated in connection with the first embodiment is preferably human. More preferably it is a human female in which a previous in vitro fertilization programme failed due to an implantation failure of the embryo. In such case and also generally the method of increasing embryonic implantation may also be a method for treating or preventing implantation failure.

The nucleic acid sequences of SEQ ID NOs 18 to 34 are the genes encoding human HLA-H, HLA-J, HLA-L soluble, HLA-L membrane-bound, HLA-V, HLA-Y, HLA-E, HLA-F1, F2, F2 and HLA-G1, G2, G3, G4, G5, G6 and G7, respectively. Among HLA-F1 to F3 the HLAs of F1 and F5 are preferred. Similarly, among HLA-G1 to G7 the HLAs of G1 and G5 are preferred.

It is reported in the application EP 19 18 4681.5 that the gene encoding HLA-L comprises a sequence encoding a transmembrane domain and also that soluble HLA-L can be detected. It is therefore believed HLA-L can be found as a full-length membrane-bound form as well as a soluble form. Full-length HLA-L might also be released by post-translational proteolytic cleavage to result in the release of soluble HLA fragments.

The primary transcript of HLA-G (8 Exons, NCBI Gene Bank NM_002127.5, version of Sep. 16, 2019) can be spliced into 7 alternative mRNAs that encode membrane-bound (HLA-G1, -G2, -G3, -G4) and soluble (HLA-G5, -G6, -G7) protein isoforms (Carosella et al., 2008, Trends Immunol.; 29(3):125-32). HLA-G1 is the full-length HLA-G molecule, HLA-G2 lacks exon 4, HLA-G3 lacks exons 4 and 5, and HLA-G4 lacks exon 5. HLA-G1 to -G4 are membrane-bound molecules due to the presence of the transmembrane and cytoplasmic tail encoded by exons 6 and 7. HLA-G5 is similar to HLA-G1 but retains intron 5, HLA-G6 lacks exon 4 but retains intron 4, and HLA-G7 lacks exon 4 but retains intron 2. HLA-G5 and -G6 are soluble forms due to the presence of intron 4, which contains a premature stop codon to prevent the translation of the transmembrane and cytoplasmic tail. HLA-G7 is soluble due to the presence of intron 3, which contains a premature stop codon. Also HLA-F is alternatively spliced. The three isoforms F1, F2 and F3 are all membrane-bound isoforms.

No alternative splice forms of HLA-H, HLA-J, HLA-V, HLA-Y, and HLA-E are known. HLA-H, HLA-J, HLA-V, HLA-Y are soluble and HLA-E is membrane-bound.

The term “nucleic acid sequence” or “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. Certain nucleic acid molecules, for example, shRNAs, miRNAs, or an antisense nucleic acid molecules as described herein below, may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., antibodies, signal peptides, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule may additionally comprise promoters, enhancers, response elements, signal sequences, polyadenylation sequences, introns, 5′- and 3′-non-coding regions, and the like.

It is preferred that the nucleic acid molecule according to the invention is genomic DNA or mRNA. In the case of mRNA, the nucleic acid molecule may in addition comprise a poly-A tail.

The amino acid sequences of SEQ ID NOs 1 to 17 are the human HLA proteins HLA-H, HLA-J, HLA-L soluble, HLA-L membrane bound, HLA-V, HLA-V, HLA-Y, HLA-E, HLA-F1, F2, F3 and HLA-G1, G2, G3, G4, G5, G6 and G7, respectively. Again, among HLA-F1 to F3 the HLAs of F1 and F2 are preferred and among HLA-G1 to G7 the HLAs of G1 and G5 are preferred.

The term “protein” as used herein interchangeably with the term “polypeptide” describes linear molecular chains of amino acids, including single chain proteins or their fragments, containing at least 50 amino acids. The term “peptide” as used herein describes a group of molecules consisting of up to 49 amino acids. The term “peptide” as used herein describes a group of molecules consisting with increased preference of at least 15 amino acids, at least 20 amino acids at least 25 amino acids, and at least 40 amino acids. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. For example, the HLA proteins comprise cysteins and thus potential dimerization sites. The terms “(poly)peptide” and “protein” also refer to naturally modified (poly)peptides and proteins where the modification is effected e.g. by glycosylation, acetylation, phosphorylation and similar modifications which are well known in the art.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment for two or more sequences or subsequences the percentage of amino acid residues or nucleotides that are the same (e.g. 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

As defined herein, sequence identities of at least 85% identity, preferably at least 90% identity, and most preferred at least 95% identity are envisaged by the invention. However, also envisaged by the invention are with increasing preference sequence identities of at least 97.5%, at least 98.5%, at least 99%, at least 99.5%, at least 99.8%, and 100% identity. In connection with all these sequences it is preferred that they retain the capability of the corresponding SEQ ID NO: of encoding a protein or peptide being capable of immunosuppression. In connection with these sequences it is also preferable that they retain being capable of increasing implantation efficacy.

In accordance with a preferred embodiment of the first aspect of the invention the nucleic acid molecule is fused to a heterologous nucleotide sequence, preferably operably linked to a heterologous promoter.

The heterologous nucleotide sequence can either be directly or indirectly fused to the nucleic acid molecule in accordance with invention. In case of an indirect fusion preferably nucleotide sequences encoding a peptide linker are used for the fusion, such that a GS-linker (e.g. Gly-Gly-Gly-Gly-Ser)n (SEQ ID NO: 35), wherein n is 1 to 3).

As used herein, a heterologous nucleotide sequence is a sequence that cannot be found in nature fused to the nucleotide sequences as defined in connection with the first aspect of the invention. Noting these nucleotide sequences are from human, it is preferred that the heterologous nucleotide sequence is also derived from human.

Accordingly, a heterologous promoter is a promoter that cannot be found in nature operably linked to the nucleotide sequence as defined in connection with the first aspect of the invention. The heterologous promoter is preferably from human.

A promoter is a nucleic acid sequence that initiates transcription of a particular gene, said gene being in accordance with the invention as defined in connection with the first aspect of the invention. In this connection “operably linked” shall mean that the heterologous promoter is fused to the nucleic acid molecule in accordance with the invention, so that via the promoter the transcription of the nucleic acid molecule in accordance with the invention can be initiated. The heterologous promoter can be a constitutively active promoter, a tissue-specific or development-stage-specific promoter, an inducible promoter, or a synthetic promoter. Constitutive promoters direct expression in virtually all tissues and are largely, if not entirely, independent of environmental and developmental factors. As their expression is normally not conditioned by endogenous factors, constitutive promoters are usually active across species and even across kingdoms. Tissue-specific or development-stage-specific promoters direct the expression of a gene in specific tissue(s) or at certain stages of development. The activity of inducible promoters is induced by the presence or absence of biotic or abiotic factors. Inducible promoters are a very powerful tool in genetic engineering because the expression of genes operably linked to them can be turned on or off as needed. Synthetic promoters are constructed by bringing together the primary elements of a promoter region from diverse origins.

Non-limiting examples of heterologous promoters which are used in the art in order to express genes heterologously are SV40, CMV, HSV, UBC, EF1A, PGK, Vlambda1, RSV and CAGG (for mammalian systems); COPIA and ACT5C (for Drosophila systems) and GAL1, GAL10, GALT, GAL2 (for yeast systems) and can also be employed in connection with the present invention.

Promoters for high and stable transgene expression in humans are, for example, described in Hoffmann et al., Gene Ther. 2017 May; 24(5):298-307. doi: 10.1038/gt.2017.20. Epub 2017 Apr. 20. Non-limiting examples of suitable promoters are the ubiquitous promoters CMV, CAG, CBA, and EF1 a and the tissue specific promoters Synapsin, CamKIIa, GFAP, RPE, ALB, TBG, MBP, MCK, TNT, and aMHC.

Alternatively or in addition, the heterologous nucleic acid sequence may be a coding sequence such that the nucleic acid sequence of the invention gives rise to a fusion protein.

The term “degenerate” as used herein refers to the degeneracy of the genetic code. The degeneracy of codons is the redundancy of the genetic code, exhibited as the multiplicity of three-base pair codon combinations that specify an amino acid. The degeneracy of the genetic code is what accounts for the existence of synonymous mutations.

The fragment comprising at least 150 nucleotides, preferably at least 300 nucleotides, more preferably at least 450 nucleotides, and most preferably at least 600 nucleotides is preferably a fragment retaining the capability of the corresponding full-length sequence of encoding a protein or peptide being capable of immunosuppression. In connection with the first aspect the fragments are also preferably capable of increasing implantation efficacy. It is most preferred that the fragment is a fragment only lacking the 5′-ATP start codon and/or the 3′-TAG stop codon.

The term “vector” in accordance with the invention means preferably a plasmid, cosmid, virus, bacteriophage or another vector used e.g. conventionally in genetic engineering which carries the nucleic acid molecule according to the invention. The nucleic acid molecule according to the invention may, for example, be inserted into several commercially available vectors. For the expression of a transgene in humans via a vector lentiviral vectors and adeno-associated vectors (AAV) are preferred and AAV is more preferred. AAV is a non-enveloped virus that can be engineered to deliver DNA to target cells, and has attracted a significant amount of attention in the field, especially in clinical-stage experimental therapeutic strategies. The ability to generate recombinant AAV particles lacking any viral genes and containing DNA sequences of interest for various therapeutic applications has thus far proven to be one of the safest strategies for gene therapies (for review, Naso et al. (2017), BioDrugs; 31(4): 317-334.).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein according to the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide according to the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically-designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules according to the invention.

The term “host cell” means any cell of any organism that is selected, modified, transformed, grown, or used or manipulated in any way, for the production of the protein or peptide or fusion protein according to the invention by the cell.

The host cell according to the invention is typically produced by introducing the nucleic acid molecule or vector(s) according to the invention into the host cell which upon its/their presence mediates the expression of the nucleic acid molecule according to the invention encoding the protein or peptide or fusion protein according to the invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli (e.g., E coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may a Saccharomyces cerevisiae cell, Pichia pastoris cell or an Aspergillus cell. Preferred examples for host cell to be genetically engineered with the nucleic acid molecule or the vector(s) according to the invention is a cell of yeast, E. coli and/or a species of the genus Bacillus (e.g., B. subtilis). In one preferred embodiment the host cell is a yeast cell (e.g. S. cerevisiae).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucell's Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

The rate at which a fertilized oocyte or preimplantation embryo successfully implants depends mostly on two factors: the quality of the embryo and the receptiveness of the uterus. With respect to the receptiveness of the uterus it is of note that during implantation the embryo (via the trophoblast) comes into direct contact with the wall of the uterus. In case the oocyte in the IVF programme is from the female to receive the fertilized oocyte or preimplantation embryo the embryo is a semi-allotransplant due to the genetic information from the father and in case the oocyte in the IVF programme is from a female donor the embryo is even an allotransplant. It is very remarkable that the uterus nevertheless does not reject the embryo but generally allows the embryo to adhere to the uterus. It is assumed that the HLA class Ib genes HLA-E, HLA-F (F1 to F3) and HLA-G (G1 to G7) as well as the HLA genes HLA-H, HLA-J, HLA-L, HLA-V and HLA-Y are expressed by the embryo and exert a predominantly immunosuppressive function which allows the embryo to become implanted without being rejected by the mother. While the expression of HLA-E, HLA-F and HLA-G in pregnancy and cancer is known from the prior art, the prior art to the best knowledge of the inventors does not describe any particular role of HLA-E, HLA-F and HLA-G for the implantation of the preimplantation embryo into the uterus, let alone in an in vitro fertilization programme.

With respect to HLA-H, HLA-J and HLA-L it was previously surprisingly found by the applicant that HLA-L, HLA-H and HLA-J were erroneously annotated as pseudogenes in the art. In fact, these genes are protein-coding and the expression of HLA-L, HLA-H and HLA-J was detected in various cancers (PCT/EP2019/060606, EP 19 18 4729.2, EP 19 18 4681.5 and EP 19 18 4717.7). Moreover, a promoter region and an open reading frame was also found in HLA-V and HLA-Y. Since HLA-L, HLA-H, HLA-J, HLA-V and HLA-Y all were erroneously annotated in the art, HLA-L, HLA-H, HLA-J, HLA-V and HLA-Y may be collectively described as new HLA-group, which is called herein class Iw. In addition, high expression level of HLA-L, HLA-H and HLA-J in patients having bladder cancer was found to be adversely associated with the survival of these patients. The higher the expression level of these HLA genes the more likely the patients died from the cancer within 2 years (EP 19 18 4681.5 and EP 19 18 4717.7). This body of evidence shows that the expression of the HLA forms L, H and J is used by tumors as a mechanism of evading the immune system of the tumor patient. The same can be assumed for HLA-V and HLA-Y. This data on cancer renders it at least plausible that during implantation HLA-L, HLA-H, HLA-J, HLA-V and HLA-Y are expressed by the embryo to evade the immune system of the mother which then allows the embryo to become implanted into the uterus.

In summary, HLA-H, HLA-J, HLA-L, HLA-V, HLA-V, HLA-Y, HLA-E, HLA-F and HLA-G increase receptiveness of the uterus for the embryo which in turn increases efficiency of embryonic implantation during an IVF programme. The supplementation of blastocyte cell culture medium with HLA class Ib and Iw genes thereby increases efficiency of embryonic implantation during an IVF programme is illustrated by Example 1. Moreover, Example 2 shows the expression of HLA-class Ib and Iw genes in blastocyte cell culture medium. Therefore, a nucleic acid molecule, a vector, a host cell, or a protein or peptide, or any combination thereof according to the invention when being used in a method as defined herein above increases the receptiveness of the uterus which in turn leads to increasing efficiency of embryonic implantation during an IVF programme.

In this connection the nucleic acid molecule, vector, host cell, or protein or peptide, or any combination thereof may be contacted with the unfertilized, fertilized oocyte, and/or preimplantation embryo prior to the transfer of the fertilized oocyte or preimplantation embryo to the uterus. In practice this may be done, for example, by culturing the unfertilized, fertilized oocyte, and/or preimplantation embryo in a culture medium comprising the nucleic acid molecule, vector, host cell, or protein or peptide, or any combination thereof. This will lead to increasing the amount of HLA-H, HLA-J, HLA-L, HLA-V, HLA-E, HLA-F and/or HLA-G in the fertilized oocyte or preimplantation embryo at the time when it is transferred to the uterus and subsequently reaches the wall of the uterus.

Also the nucleic acid molecule, vector, host cell, or protein or peptide, or any combination thereof may be contacted with the uterus prior to, simultaneously with and/or after the transfer of the fertilized oocyte or preimplantation embryo to the uterus. In practice this may be done, for example, by preparing a solution comprising the nucleic acid molecule, vector, host cell, or protein or peptide, or any combination thereof for rinsing the uterus prior to, simultaneously with and/or after the transfer of the fertilized oocyte or preimplantation embryo to the uterus. This approach likewise leads to increasing the amount of HLA-H, HLA-J, HLA-L, HLA-V, HLA-E, HLA-F and/or HLA-G at the interface of the fertilized oocyte or preimplantation embryo and the uterus at the time when the implantation takes place.

As a yet further alternative, the nucleic acid molecule, vector, host cell, or protein or peptide, or any combination may be systemically administered prior to, simultaneously with and/or after the transfer of the fertilized oocyte or preimplantation embryo to the uterus, preferably via injection, transdermal and/or vaginal administration. Also the systemic administration will lead to increasing the amount of HLA-H, HLA-J, HLA-L, HLA-V, HLA-E, HLA-F and/or HLA-G at the interface of the fertilized oocyte or preimplantation embryo and the uterus at the time when the implantation takes place.

In accordance with a preferred embodiment of the first aspect of the invention prior to in vitro fertilization is (i) any time between after the collection of the unfertilized oocyte and directly before the transfer of the fertilized oocyte or preimplantation embryo to the uterus, and (ii) preferably any time between after the fertilization of the oocyte by sperm and directly before the transfer of the fertilized oocyte or preimplantation embryo to the uterus.

As discussed herein above, the IVF programme comprises (i) collecting a mature unfertilized oocyte from ovaries of a donor female, (ii) fertilization of the mature unfertilized oocyte with sperm to obtain a fertilized ooyte, (iii) optionally in vitro preimplantation development of the fertilized ooyte into a preimplantation embryo, and (iv) transfer of the fertilized ooyte or a preimplantation embryo to the uterus of a female.

In accordance with the above preferred embodiment prior to in vitro fertilization can be at stages (ii) or (iii) and preferably is at stage (iii).

In accordance with a further preferred embodiment of the first aspect of the invention after in vitro fertilization is (i) any time between directly after the transfer of the fertilized oocyte or preimplantation embryo to the uterus and 6 days after the transfer of the fertilized oocyte or preimplantation embryo to the uterus, (ii) preferably is any time between directly after the transfer of the fertilized oocyte or preimplantation embryo to the uterus and 4 days after the transfer of the fertilized oocyte or preimplantation embryo to the uterus, and (iii) most preferably is any time between directly after the transfer of the fertilized oocyte or preimplantation embryo to the uterus and 2 days after the transfer of the fertilized oocyte or preimplantation embryo to the uterus.

With respect to this preferred embodiment it is of note that the transfer of the fertilized oocyte or the preimplantation embryo can take place in humans any time directly after the fertilization of the oocyte and up to day six after the fertilization of the oocyte. This is because after fertilization the blastocyste hatches at day six and is ready to implant. If the blastocyste hatches outside the uterus it can no longer implant into the uterus.

The invention relates in a second aspect to an ex vivo or in vitro method for increasing the likelihood of a fertilized oocyte or preimplantation embryo to become implanted during in vitro fertilization of a female comprising culturing an isolated oocyte or preimplantation embryo in the presence of a nucleic acid molecule, a vector, a host cell, or a protein or peptide, or any combination thereof as defined in the first aspect of the invention.

The definitions and preferred embodiments of the first aspect of the invention apply mutatis mutandis to the second aspect of the invention.

The IVF steps of the IVF programme of (i) to (iv) have been discussed herein above. The ex vivo or in vitro method of the second aspect does not comprise the in vivo steps (i) and (iv) but relates to the ex vivo or in vitro production of a fertilized oocyte or preimplantation embryo having an increased likelihood to become implanted during in vitro fertilization (when subsequently being used in step (iv)) as compared to an isolated oocyte or preimplantation embryo that has been cultured in the absence of a nucleic acid molecule, a vector, a host cell, or a protein or peptide, or any combination thereof as defined in the first aspect of the invention.

In accordance with a preferred embodiment of the second aspect of the invention the isolated oocyte is an unfertilized oocyte or a fertilized oocyte.

Hence, the isolated oocyte can be cultured in the presence of a nucleic acid molecule, a vector, a host cell, or a protein or peptide, or any combination thereof before and/or after it has been fertilized with sperm.

The invention relates in a third aspect to a nucleic acid molecule, a vector, a host cell, or a protein or peptide, or any combination thereof as defined in the first aspect of the invention for use in preventing abortion during pregnancy.

The definitions and preferred embodiments of the above aspects of the invention apply mutatis mutandis to the third aspect of the invention.

Likewise, the invention relates to a method for preventing abortion during pregnancy comprising administering to a pregnant female a nucleic acid molecule, a vector, a host cell, or a protein or peptide, or any combination thereof as defined in the first aspect of the invention.

Abortion is the ending of a pregnancy by expulsion of an embryo or foetus before it can survive outside the uterus. The abortion in accordance with the third aspect is to occur without intervention and may also be designated miscarriage or spontaneous abortion. Between 15% and 30% of known pregnancies end in clinically apparent miscarriage, depending upon the age and health of the pregnant woman. 80% of these spontaneous abortions happen in the first trimester.

Also after the embryo has become implanted to the uterus the embryo may still be rejected by the mother. Such a rejection reaction may be prevented by the immunosuppressive effect of HLA-H, HLA-J, HLA-L, HLA-V, HLA-E, HLA-F and/or HLA-G. Therefore, a nucleic acid molecule, a vector, a host cell, or a protein or peptide, or any combination thereof according to the invention can also be used to prevent abortion.

The invention relates in a fourth aspect to a nucleic acid molecule, a vector, a host cell, or a protein or peptide, or any combination thereof as defined in the first aspect of the invention for use in treating or preventing pre-eclampsia in a pregnant female.

The definitions and preferred embodiments of the above aspects of the invention apply mutatis mutandis to the fourth aspect of the invention.

Likewise, the invention relates to a method for treating or preventing pre-eclampsia in a pregnant female comprising administering to the pregnant female a nucleic acid molecule, a vector, a host cell, or a protein or peptide, or any combination thereof as defined in the first aspect of the invention.

Pre-eclampsia is a condition that affects some pregnant women, usually during the second half of pregnancy (from around 20 weeks) or soon after their baby is delivered. Mild pre-eclampsia affects up to 6% of pregnancies, and severe cases develop in about 1 to 2% of pregnancies. Pre-eclampsia is characterized by high blood pressure and signs of damage to another organ system, most often the liver and kidneys. Left untreated, preeclampsia can lead to serious and even fatal complications for both the mother and the foetus/baby.

Currently, the most effective treatment is delivery of the baby although even after delivering the baby, it can still take a while until the symptoms disappear.

Pre-eclampsia begins in the placenta, being the organ that nourishes the foetus throughout pregnancy. Early in pregnancy, new blood vessels develop and evolve to efficiently send blood to the placenta.

Trophoblasts are the cells forming the outer layer of a blastocyst, which provide nutrients to the embryo and develop into a large part of the placenta. A variety of immune cells and immune effector molecules are found in the feto-maternal interface and have been described as important players for maintaining immune tolerance toward the semi-allogeneic foetus. It is assumed that the fetal trophoblast cells express HLA-H, HLA-J, HLA-L, HLA-V, HLA-E, HLA-F and/or HLA-G and by their immunosuppressive effect are protected from maternal T cell-mediated alloreactivity and that the maternal T cell-mediated alloreactivity is involved in pre-eclampsia.

It follows that a nucleic acid molecule, a vector, a host cell, or a protein or peptide, or any combination thereof according to the invention can also be used to treat or prevent pre-eclampsia.

In accordance with a preferred embodiment of the fourth aspect of the invention the pregnant female carries a male embryo or foetus.

The prevalence of pre-eclampsia is higher in women carrying a male embryo or foetus than in women carrying a female embryo or foetus. The reason is presumably that the male foetus is more alloreactive due to the presence of the allogeneic Y chromosome.

The invention relates in a fifth aspect to an inhibitor of the nucleic acid molecule or a protein or peptide as defined in the first aspect of the invention for use in treating or preventing the HELLP syndrome.

The definitions and preferred embodiments of the above aspects of the invention apply mutatis mutandis to the fifth aspect of the invention.

Likewise, the invention relates to a method for treating or preventing the HELLP syndrome comprising administering to a pregnant female or a female that gave birth in need thereof an inhibitor of the nucleic acid molecule or a protein or peptide as defined in the first aspect of the invention.

HELLP syndrome (hemolysis, elevated liver enzymes, and low platelets)(hemolysis, elevated liver enzymes, and low platelets)(hemolysis, elevated liver enzymes, and low plat is a complication of pregnancy characterized by hemolysis, elevated liver enzymes, and a low platelet count. It usually begins during the last three months of pregnancy or shortly after childbirth. HELLP syndrome occurs in about 0.7% of pregnancies. In HELLP syndrome cells of the foetus flood the organism of the mother and lead to a graft vs. host reaction in the mother. The HELLP syndrome is more frequent in the first pregnancy since the immune system of the mother is not yet tolerized against an embryo.

In order to enable the immune system of the mother to recognize the foetal cells floating the body of the mother an inhibitor of the nucleic acid molecule or a protein or as defined in the first aspect of the invention may be used. Such an inhibitor suppresses the immunotolerance of the immune system of the mother against the foetal cells, since the immunosuppressive effect of HLA-H, HLA-J, HLA-L, HLA-V, HLA-E, HLA-F and/or HLA-G is suppressed by the inhibitor.

In accordance with a preferred embodiment of the fifth aspect of the invention (i) the inhibitor of the nucleic acid molecule is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (ii) the binding molecule of the protein, preferably the inhibitor of the protein is selected from a small molecule, an antibody or antibody mimetic, an aptamer.

In accordance with a more preferred embodiment of the fifth aspect of the invention the antibody mimetic is preferably selected from affibodies, adnectins, anticalins, DARPins, avimers, nanofitins, affilins, Kunitz domain peptides, Fynomers®, trispecific binding molecules and probodies.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about Da amu. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

The term “antibody” as used in accordance with the present invention comprises, for example, polyclonal or monoclonal antibodies. Furthermore, also derivatives or fragments thereof, which still retain the binding specificity to the target, e.g. the HLA-J, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler EP, Serebryanaya DV, Katrukha AG. 2010, Biochemistry (Mosc)., vol. 75(13), 1584; Holliger P, Hudson PJ. 2005, Nat Biotechnol., vol. 23(9), 1126). The multimeric formats in particular comprise bispecific antibodies that can simultaneously bind to two different types of antigen. The first antigen can be found on the protein in accordance with the invention. The second antigen may, for example, be a placenta marker that is specifically expressed on trophoblast cells or a certain type of cells the uterus. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847). The use of such bispecific antibodies allows focusing the action of the bispecific antibodies, for example; to the uterus or placenta. Placental biomarkers are, for example, described in Manokhina et al. (2017), Hum Mol Genet.; 26(R2):R237-R245.

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson PJ. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope, for example, of HLA-J. Surface plasmon resonance as employed in the BIAcore system can be used to increase the efficiency of phage antibodies.

As used herein, the term “antibody mimetics” refers to compounds which, like antibodies, can specifically bind antigens, such the HLA-J protein, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, e.g. against HLA-J, can be genetically engineered by introducing modifications in specific loops of the protein.

The term “anticalin”, as used herein, refers to an engineered protein derived from a lipocalin (Beste G, Schmidt FS, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, e.g. for HLA-J, can be selected, for example, by phage display techniques. The binding specificity of the different A-domains contained in an avimer may, but does not have to be identical (Weidle UH, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. HLA-J, by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, e.g. against HLA-J, is effected, for example, by phage display or ribosome display techniques. Depending on the scaffold, affilins have a molecular weight of approximately 10 or 20 kDa. As used herein, the term affilin also refers to di- or multimerised forms of affilins (Weidle UH, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6kDA and domains with the required target specificity, e.g. against HLA-J, can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein of the fourth aspect of the invention. The two other epitopes may also be epitopes of the protein of the fourth aspect of the invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop (SEQ ID NO: 36) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

As used herein, the term “probody” refers to a protease-activatable prodrug, e.g. to a protease-activatable antibody prodrug. A probody, for example, consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects. It is furthermore possible to confine the binding and/or inhibitory activity of the small molecule, antibody or antibody mimetic and aptamer to certain tissues or cell-types, in particular diseased tissues or cell-types by probodies. In such a probody the small molecule, antibody or antibody mimetic or aptamer is also bound to a masking peptide which limits or prevents binding to the protein of the invention and which masking peptide can be cleaved by a protease. Proteases are enzymes that digest proteins into smaller pieces by cleaving specific amino acid sequences known as substrates. In normal healthy tissue, protease activity is tightly controlled. In cancer cells, protease activity is upregulated. In healthy tissue or cells, where protease activity is regulated and minimal, the target-binding region of the probody remains masked and is thus unable to bind. On the other hand, in diseased tissue or cells, where protease activity is upregulated, the target-binding region of the probody gets unmasked and is thus able to bind and/or inhibit.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor (for example, of HLA-J) after introduction into the respective cells.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here, for example, the HLA-J gene. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls, for example, HLA-J expression. Alternatively, the DNA-binding domain of zinc finger nucleases (ZFNs) or transcription activator-like effector nucleases (TALENs) can be designed to specifically recognize the target (e.g. HLA-J) gene or its promoter region or its 5′-UTR thereby inhibiting the expression of the target.

Inhibitors provided as inhibiting nucleic acid molecules that target the gene of interest or a regulatory molecule involved in its expression are also envisaged herein. Such molecules, which reduce or abolish the expression of the target gene or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27; Miller et al., Nature biotechnology. 2011; 29(2):143-148, and Klug, Annual review of biochemistry. 2010; 79:213-231.

The invention relates in a sixth aspect to a nucleic acid molecule, a vector, a host cell, or a protein or peptide, or any combination thereof as defined in the first aspect for use in treating or preventing an autoimmune disease, preferably in a pregnant female, wherein the autoimmune disease is preferably dermatomyositis, Hashimoto's thyroiditis, Sjögren syndrome or sclerodermia.

The definitions and preferred embodiments of the above aspects of the invention apply mutatis mutandis to the sixth aspect of the invention.

Similarly, the invention relates to a method of treating an autoimmune disease comprising administering a nucleic acid molecule, a vector, a host cell, or a protein or peptide, or any combination thereof as defined in the first aspect to the subject to be treated which is preferably in a pregnant female.

An autoimmune disease is a condition in which the own immune system of a subject attacks the body of the subject. The immunosuppressive effect of HLA-H, HLA-J, HLA-L, HLA-V, HLA-V, HLA-Y, HLA-E, HLA-F and HLA-G is expected prevent or treat the unwanted immune reaction to the body of the subject. Therefore, a nucleic acid molecule, a vector, a host cell, or a protein or peptide, or any combination thereof according to the invention is a means for treating an autoimmune disease.

The autoimmune disease is preferably selected from dermatomyositis, Hashimoto's thyroiditis, Sjögren syndrome and sclerodermia, noting that these autoimmune diseases have a higher prevalence in pregnant women as compared to non-pregnant women.

The invention relates in a seventh aspect to a nucleic acid molecule, a vector, a host cell, or a protein or peptide, or any combination thereof as defined in the first aspect for use in treating or preventing graft versus host disease.

The definitions and preferred embodiments of the above aspects of the invention apply mutatis mutandis to the seventh aspect of the invention.

Also the invention relates to a method of treating or preventing graft versus host disease comprising administering to a subject a nucleic acid molecule, a vector, a host cell, or a protein or peptide, or any combination thereof as defined in the first aspect.

Graft-versus-host disease (GVHD) is an immune condition that occurs in a patient after transplantation when immune cells present in donor tissue (the graft) attack the host's own tissues. GVHD is a complication after bone marrow transplantation (stem cell transplants) from both related and unrelated donors. These types of transplants are called allogeneic transplants. With acute GVHD and chronic GVHD, symptoms can range from mild to severe and life-threatening and often include skin inflammation, jaundice, and gastrointestinal (GI) discomfort along with other organ problems. Acute GVHD usually occurs within the first 100 days post-transplant; the acute form of the disease causes clinical symptoms of skin rash, liver problems, and intestinal symptoms like nausea and diarrhea. Chronic GVHD occurs later; the chronic form of the disease may affect a number of different organs and body systems. GVHD has a complex pathophysiology that involves a number of interactions between the immune cells of the transplant donor and recipient patient.

The immunosuppressive effect of HLA-H, HLA-J, HLA-L, HLA-V, HLA-V, HLA-Y, HLA-E, HLA-F and HLA-G is expected to prevent or treat GVHD. Therefore, a nucleic acid molecule, a vector, a host cell, or a protein or peptide, or any combination thereof according to the invention is a means for treating an GVHD.

Finally, in accordance with a preferred embodiment of all previous aspects of the invention (I) the at least one nucleic acid molecule is selected from nucleic acid molecules (a) encoding a polypeptide comprising or consisting of the amino acid sequence of any one of SEQ ID NOs 1 to 6, (b) comprising or consisting of the nucleotide sequence of any one of SEQ ID NOs 18 to 23, (c) encoding a polypeptide which is at least 85% identical, preferably at least 90% identical, and most preferred at least 95% identical to the amino acid sequence of (a), (d) consisting of a nucleotide sequence which is at least 95% identical, preferably at least 96% identical, and most preferred at least 98% identical to the nucleotide sequence of (b), (e) consisting of a nucleotide sequence which is degenerate with respect to the nucleic acid molecule of (d), (f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and (g) corresponding to the nucleic acid molecule of any one of (a) to (f), wherein T is replaced by U, and (II) the vector comprises the nucleic acid molecule of (I); (III) the host cell is transformed, transduced or transfected with the vector of (II); and (IV) the at least one protein or peptide is selected from proteins or peptides being encoded by the nucleic acid molecule of (I).

With respect to the fifth aspect of the invention which reads on an inhibitor of the nucleic acid molecule or a protein or peptide as defined in the first aspect of the invention for the treatment of the HELLP syndrome it is to be understood that this inhibitor is preferably an inhibitor of the nucleic acid molecule nucleic acid molecule as defined in (I) or of the protein or peptide as defined in (IV) of the above preferred embodiment.

SEQ ID NOs 1 to 6 are the amino acid sequences of HLA-H, HLA-J, HLA-L soluble, HLA-L membrane bound, HLA-V, HLA-V, and HLA-Y and SEQ ID NO 18 to 23 are the nucleotide sequences encoding HLA-H, HLA-J, HLA-L, soluble, HLA-L membrane bound, HLA-V, HLA-V, and HLA-Y. As discussed herein above, it is the contribution of the present application as well as the herein above cited previous applications of the applicant that HLA-H, HLA-J, HLA-L, soluble, HLA-L membrane bound, HLA-V, HLA-V, HLA-Y were previously mischaracterized as pseudogenes but are in fact protein-coding genes. For the reasons provided herein above HLA-H, HLA-J, HLA-L, soluble, HLA-L membrane bound, HLA-V, HLA-V, HLA-Y or inhibitors thereof are useful for the various medical treatments as disclosed herein as well as method of the second aspect of the invention. With respect to HLA-L the soluble form is preferred as compared to the membrane bound form.

In connection with the above preferred embodiment it is preferred to use in addition HLA-G, HLA-E and/or HLA-F or inhibitor thereof, depending on whether the medical use requires promoting or inhibiting the immuntolergenic effect of these HLAs.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The examples illustrate the invention.

### EXAMPLE 1: SUPPLEMENTATION OF BLASTOCYTE CELL CULTURE MEDIUM WITH HLA CLASS IB AND IW GENES

This example relates to a procedure to support implantation of an in vitro fertilized embryo by supplementing blastocyte cell culture with synthesised HLA class Ib and Iw molecules or by applying them systemically.

HLA-G, HLA-H, HLA-J, HLA-L, HLA-V, HLA-V, HLA-Y, HLA-E, and/or HLA-F was added to embryo cell culture. The doses of HLA proteins added to the cell culture was evaluated according to Sjöblom et al. (Sjöblom C1, Wikland M, Robertson SA., Hum Reprod. 1999 December; 14(12):3069-76) and Bhatnagar et al. (Bhatnagar P1, Papaioannou VE, Biggers JD, Development. 1995 May; 121(5):1333-9). Recombinant HLA-G or HLA-H, HLA-J, HLA-L, HLA-V, HLA-V, HLA-Y, HLA-E, HLA-F proteins were generated as state of the art and according to Favier et al (Favier et al., PLoS One. 2011; 6(7): e21011).

For intravenous applications, recombinant HLA-G, HLA-H, HLA-J, HLA-L, HLA-V, HLA-V, HLA-Y, HLA-E, and/or HLA-F proteins were generated under cGMP conditions. The quality, sterility, endotoxin testing as well as chemical purity are tested according to the monographs from the European Pharmacopeia, V.8.0 (European Pharmacopoeia (Ph. Eur.) Vol 8 (2013-2016) European Directorate of Quality of Medicines).

In order to compare the effect of the HLA supplementation to implantation and pregnancy rates, a control group was also monitored which did not receive HLA protein supplementation.

The embryos were transferred at day 5, according to guidelines of the American society for reproductive medicine (Fertil Steril, 2017; 107:882-96). Successful implantation was monitored by measurement of serum levels of free beta-human Choriongonadotropin (β-hCG) and vaginal ultra sound observation.

It could be observed that implantation rates, as well as successful pregnancies were significantly higher from embryos receiving the HLA supplementation treatment as compared to the control group.

### EXAMPLE 2—EXTRACTION AND DETERMINATION OF HLA-CLASS IB AND IW GENES IN BLASTOCYTE CELL culture medium

In order to evaluate HLA class Ib and Iw expression via quantitative real-time polymerase chain reaction (qRT-PCR) from blastocyte cell culture media RNA has been extracted first. Since blastocytes shed RNA in extracellular vesicles (EVs) (Giacomini et al., Sci Rep. 2017; 7: 5210), RNA was isolated by first collecting EVs in ExoQuick™ (SBI Systems Bioscience Inc. Mountain View Calif., USA) according to protocol from König et al. (König et al., Hum Immunol. 2016 September;77(9):791-9). After the removal of the ExoQuick™ reagent, RNA was extracted according to the protocol from Stratifyer Blood extraction kit. In brief, after proteinase K digestion, lysates were admixed with germanium-coated magnetic particles in the presence of special buffers, which promote the binding of nucleic acids. Purification was carried out by means of consecutive cycles of mixing, magnetization, centrifugation and removal of contaminants. RNA was eluated with 25 μl elution buffer and RNA eluates were then stored at −80° C. until use. All extracts were tested for sufficient high-quality RNA content by quantification with qRT-PCR of the constitutively expressed gene Calmodulin 2 gene (CALM2) which is known as a stable reference/housekeeper gene. For a detailed analysis of gene expression by qRT-PCR methods, primers flanking the region of interest and a fluorescently labeled probe hybridizing in-between were used. RNA-specific primer/probe sequences were used to enable RNA-specific measurements by locating primer/probe sequences across exon/exon boundaries. In case multiple isoforms of the same gene existed, primers were selected to amplify all relevant or selected splice variants as appropriate. All primer pairs were checked for specificity by conventional PCR reactions. Specific primers have been generated against HLA-H, HLA-J, HLA-L, HLA-V, HLA-V, HLA-Y, HLA-E, HLA-F and HLA-G.

HLA-H, HLA-J, HLA-L, HLA-V, HLA-V, HLA-Y, HLA-E, HLA-F and HLA-G were found to be expressed in blastocyte cell culture.

1. A nucleic acid molecule, a vector, a host cell, or a protein or peptide, or any combination thereof for use in a method of increasing efficiency of embryonic implantation in an in vitro fertilization programme,
(I) wherein the at least one nucleic acid molecule is selected from nucleic acid molecules
(a) encoding a polypeptide comprising or consisting of the amino acid sequence of any one of SEQ ID NOs 1 to 17,
(b) comprising or consisting of the nucleotide sequence of any one of SEQ ID NOs 18 to 34,
(c) encoding a polypeptide which is at least 85% identical, preferably at least 90% identical, and most preferred at least 95% identical to the amino acid sequence of (a),
(d) consisting of a nucleotide sequence which is at least 95% identical, preferably at least 96% identical, and most preferred at least 98% identical to the nucleotide sequence of (b),
(e) consisting of a nucleotide sequence which is degenerate with respect to the nucleic acid molecule of (d),
(f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and
(g) corresponding to the nucleic acid molecule of any one of (a) to (f), wherein T is replaced by U, and
(II) the vector comprises the nucleic acid molecule of (I);
(III) the host cell is transformed, transduced or transfected with the vector of (II); and
(IV) the at least one protein or peptide is selected from proteins or peptides being encoded by the nucleic acid molecule of (I); and
wherein the method of increasing embryonic implantation efficiency comprises
(i) contacting the nucleic acid molecule, vector, host cell, or protein or peptide, or any combination thereof with the unfertilized, fertilized oocyte, and/or preimplantation embryo prior to the transfer of the fertilized oocyte or preimplantation embryo to the uterus; or
(ii) contacting the nucleic acid molecule, vector, host cell, or protein or peptide, or any combination thereof with the uterus prior to, simultaneously with and/or after the transfer of the fertilized oocyte or preimplantation embryo to the uterus; or
(iii) systemically administering the nucleic acid molecule, vector, host cell, or protein or peptide, or any combination prior to, simultaneously with and/or after the transfer of the fertilized oocyte or preimplantation embryo to the uterus, preferably via injection, transdermal and/or vaginal administration.

2. The nucleic acid molecule, vector, host cell, or protein or peptide, or any combination thereof for use according to claim 1, wherein prior to in vitro fertilization is
(i) any time between after the collection of the unfertilized oocyte and directly before the transfer of the fertilized oocyte or preimplantation embryo to the uterus, and
(ii) preferably any time between after the fertilization of the oocyte by sperm and directly before the transfer of the fertilized oocyte or preimplantation embryo to the uterus.

3. The nucleic acid molecule, vector, host cell, or protein or peptide, or any combination thereof for use according to claim 1, wherein after in vitro fertilization is
(i) any time between directly after the transfer of the fertilized oocyte or preimplantation embryo to the uterus and 6 days after the transfer of the fertilized oocyte or preimplantation embryo to the uterus,
(ii) preferably is any time between directly after the transfer of the fertilized oocyte or preimplantation embryo to the uterus and 4 days after the transfer of the fertilized oocyte or preimplantation embryo to the uterus, and
(iii) most preferably is any time between directly after the transfer of the fertilized oocyte or preimplantation embryo to the uterus and 2 days after the transfer of the fertilized oocyte or preimplantation embryo to the uterus.

4. An ex vivo or in vitro method for increasing the likelihood of a fertilized oocyte or preimplantation embryo to become implanted during in vitro fertilization of a female comprising culturing an isolated oocyte or preimplantation embryo in the presence of a nucleic acid molecule, a vector, a host cell, or a protein or peptide, or any combination thereof as defined in claim 1.

5. The method of claim 4, wherein the isolated oocyte is an unfertilized oocyte or a fertilized oocyte.

6. A nucleic acid molecule, a vector, a host cell, or a protein or peptide, or any combination thereof as defined in claim 1 for use in preventing abortion during pregnancy.

7. A nucleic acid molecule, a vector, a host cell, or a protein or peptide, or any combination thereof as defined in claim 1 for use in treating or preventing pre-eclampsia in a pregnant female.

8. The nucleic acid molecule, vector, host cell, or protein or peptide, or any combination thereof for use of claim 7, wherein the pregnant female carries a male embryo or foetus.

9. An inhibitor of the nucleic acid molecule or a protein or as defined in claim 1 for use in treating or preventing the HELLP syndrome.

10. The inhibitor for use of claim 9, wherein
(i) the inhibitor of the nucleic acid molecule is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(ii) the binding molecule of the protein, preferably the inhibitor of the protein is selected from a small molecule, an antibody or antibody mimetic, an aptamer.

11. The inhibitor for use of claim 10, wherein the antibody mimetic is preferably selected from affibodies, adnectins, anticalins, DARPins, avimers, nanofitins, affilins, Kunitz domain peptides, Fynomers®, trispecific binding molecules and probodies.

12. A nucleic acid molecule, a vector, a host cell, or a protein or peptide, or any combination thereof as defined in claim 1 for use in treating or preventing an autoimmune disease, preferably in a pregnant female, wherein the autoimmune disease is preferably dermatomyositis, Hashimoto's thyroiditis, Sjögren syndrome or sclerodermia.

13. A nucleic acid molecule, a vector, a host cell, or a protein or peptide, or any combination thereof as defined in claim 1 for use in treating or preventing graft versus host disease.

14. The inhibitor for use or the method of any preceding claim,
(I) wherein the at least one nucleic acid molecule is selected from nucleic acid molecules
(a) encoding a polypeptide comprising or consisting of the amino acid sequence of any one of SEQ ID NOs 1 to 6,
(b) comprising or consisting of the nucleotide sequence of any one of SEQ ID NOs 18 to 23,
(c) encoding a polypeptide which is at least 85% identical, preferably at least 90% identical, and most preferred at least 95% identical to the amino acid sequence of (a),
(d) consisting of a nucleotide sequence which is at least 95% identical, preferably at least 96% identical, and most preferred at least 98% identical to the nucleotide sequence of (b),
(e) consisting of a nucleotide sequence which is degenerate with respect to the nucleic acid molecule of (d),
(f) consisting of a fragment of the nucleic acid molecule of any one of (a) to (e), said fragment comprising at least 150 nucleotides, preferably at least 300 nucleotides, more preferably at least 450 nucleotides, and most preferably at least 600 nucleotides, and
(g) corresponding to the nucleic acid molecule of any one of (a) to (f), wherein T is replaced by U, and
(II) the vector comprises the nucleic acid molecule of (I);
(III) the host cell is transformed, transduced or transfected with the vector of (II); and
(IV) the at least one protein or peptide is selected from proteins or peptides being encoded by the nucleic acid molecule of (I).
